(19) United States
(12) Patent Application Publication
(10) Pub. No.: US 2007/0014764 A1
(43) Pub. Date: Jan. 18, 2007

(54) Reduction in myocardial infarction size

(76) Inventors: Andrew Levy, Kiryat Shmuel (IL); Noah Berkowitz, New Rochelle, NY (US)

Correspondence Address: PEARL COHEN ZEDEK, LLP;PEARL COHEN ZEDEK LATZER, LLP, 1500 BROADWAY 12TH FLOOR, NEW YORK, NY, 10036, US

(21) Appl. No.: 11/183,251

(22) Filed: Jul. 18, 2005

(51) Int. Cl. A61K 38/20; A61K 38/44; A61K 38/17

(52) U.S. Cl. 424/85.2; 514/12; 424/94.4

(57) ABSTRACT

This invention provides methods and compositions used for reducing the Myocaidial Infarct (MI) size in diabetic subjects exhibiting the haptoglobin (Hp) 2 allele. Specifically, the invention relates to reduction of MI in diabetic subjects carrying the Hp-2 allele by reducing the oxidative sterss in these subjects following ischemia-reperfusion injury.

### FIELD OF INVENTION

This invention relates to methods and compositions used for treating vascular complications in diabetic subjects exhibiting the haptoglobin (Hp) 2. allele. Specifically, the invention relates to reduction of Myocardial Infarct (MI) in diabetic subjects carrying the Hp-2 allele by reducing the oxidative sterss in these subjects following ischemia-reperfusion injury.

### BACKGROUND OF THE INVENTION

Despite recent advances, cardiovascular disease continues to be the leading cause of death among subjects with diabetes. Diabetes-related heart disease makes up the majority of the cardiovascular morbidity and mortality and this pathology results from synergistic interaction amongst various overlapping mechanisms. Diabetes-related heart disease is characterized by a propensity to develop premature, diffuse atherosclerotic disease, structural and functional abnormalities of the microvasculature, autonomic dysfunction and intrinsic myocardial dysfunction (the so-called diabetic ‘cardiomyopathy’, a reversible cardiomyopathy in diabetics that occurs in the absence of coronary atherosclerosis), all of which are exacerbated by hypertension and diabetic nephropathy. As far as the probability of the occurrence of an infarction is concerned, the risk for a diabetic is the same as that for a non-diabetic with a previous infarction.

Subjects with diabetes exhibiting acute myocardial infarction (MI) have an increased rate of death and heart failure. This poorer prognosis after MI in diabetic individuals appears to be due in large part to an increase in MI size. Ischemia-reperfusion plays an important role in determining the amount of injury occurring with MI. Animal models of MI have demonstrated that the injury associated with ischemia-reperfusion is markedly exaggerated in the diabetic state. The increased oxidative stress characteristic of the diabetic state is compounded during the ischemia-reperfusion process resulting in the increased generation of highly reactive oxygen species which can mediate myocardial damage both directly and indirectly by promoting an exaggerated inflammatory reaction. Functional polymorphisms in genes that modulate oxidative stress and the inflammatory response may therefore be of heightened importance in determining infarct size in the diabetic state.

### SUMMARY OF THE INVENTION

In one embodiment the invention provides a method for treatment of a cardiovascular complication in a subject having the Hp-2 allele, comprising administering to said subject an effective amount of a compound, thereby reducing oxidative stress in said subject.

In another embodiment, the invention provides a method of assessing the risk of developing large size myocardial infarction following ischemia reperfusion injury in a diabetic subject, comprising analyzing the Hp phenotype in said subject, wherein Hp 2 allele indicates a high risk of developing increased size myocardial infarct (MI).

In one embodiment, the invention provides a composition for reducing the myocardial infarct in a diabetic subject carrying the Hp 2 allele, comprising: glutathione peroxidase, an isomer, a functional derivative, a synthetic analog, a pharmaceutically acceptable salt or a combination thereof and a pharmaceutically acceptable carrier, excipient, flow agent, processing aid, a diluent or a combination thereof.

### BRIEF DESCRIPTION OF THE DRAWINGS

FIG. 1 shows quantitative image analysis of infarct size. Transverse section (15 μm) of the left ventricle from mouse heart post ischemia-reperfusion procedure at 50× magnification. The area of myocardial necrosis (infarct size) is stained deep red by propidium iodide. Endothelial cells from the area not at risk are stained blue with thioflavin-S Area at risk is defined as the non-blue stained area. Picture analysis was automated using pixel color coordinates (color intensity) which were the same for all sections.

FIG. 2 shows time course of I1-10 released from human PBMCs in response to 250 ug/ml Hp-Hb complex. Conditioned media was collected at 2, 5, 10 and 20 hours after treatment with the Hp-Hb complex and I1-10 measured by ELISA. Each data point represents the mean of 6. independent measurements ± SME. There was a statistically significant increase in I1-10 release in Hp 1-1-Hb treated PBMCs as compared to Hp 2-2-Hb treated PBMCs at each of the time points shown.

FIG. 3 shows dose response curve of I1-10. release from PBMCs by the Hp-Hb complex I1-10 (note log scale) was measured by ELISA 18. hours after the addition of the complex. Values shown represent the increase in I1-10 as compared to cells which were not exposed to Hp-Hb during the incubation period (mean 36±2 pg). Values shown represent the mean ± SME of 6 different measurements.

### DETAILED DESCRIPTION OF THE INVENTION

Reactive oxygen species and inflammation play critical roles in the myocardial injury associated with ischemia-reperfusion. In the cellular environment of Diabetes Melitus (DM), these processes appear to be markedly exacerbated due to the increased oxidative stress and inflammatory cytokine production associated with the hyperglycemic state. Accordingly, genetic differences in protection from oxidative stress and inflammation are expected to be important in determining infarct size after ischemia-reperlfusion injury

Haptoglobin (Hp) is a highly conserved plasma glycoprotein and is the major protein that binds free hemoglobin (Hb) with a high avidity (kd, ˜1×10⁻¹⁵ mol/L). Ischemia-reperfusion is associated with intravascular hemolysis and hemoglobin (Hb) release into the bloodstream. Extracorpuscular hemoglobin (Hb) is rapidly bound by Hp. The role of the Hp-Hb complex in modulating oxidative stress and inflammation after ischemia-reperfusion is Hp genotype dependent.

Haptoglobin is inherited by two co-dominant autosomal alleles situated on chromosome 16 in humans, these are Hp1 and Hp2 . There are three phenotypes Hp1-1, Hp2-1 and Hp2-2. Haptoglobin molecule is a tetramer comprising of four polypeptide chains, two alpha and two beta chains, of which alpha chain is responsible for polymorphism because it exists in two forms, alpha-1 and alpha-2. Hp1-1 is a combination of two alpha-1 chains along with two beta chains. Hp2-1 is a combination of one α-1 chain and one alpha-2. chain along with two beta chains. Hp2-2 is a combination of two α-2 chains and two beta chains Hp1-1 individuals have greater hemoglobin binding capacity when compared to those individuals with Hp2-1 and Hp2-2.

Hp in subjects with the Hp 1-1 phenotype is able to bind more hemoglobin on a Molar basis than Hps containing products of the haptoglobin 2. allele. Haptoglobin molecules in subjects with the haptoglobin 1-1 phenotype are also more efficient antioxidants, since the smaller size of haptoglobin 1-1 facilitates in one embodiment, its entry to extravascular sites of oxidative tissue injury compared to products of the haptoglobin 2 allele. In another embodiment, this also includes a significantly greater glomerular sieving of haptoglobin in subjects with Hp- 1-1 phenotype.

The gene differentiation to Hp-2 from Hp-1 resulted in a dramatic change in the biophysical and biochemical properties of the haptoglobin protein encoded by each of the 2 alleles. The haptoglobin phenotype of any individual, 1-1, 2-1 or 2-2, is readily determined in one embodiment, from 10 μl of plasma by gel electrophoresis.

Haptoglobin phenotype is predictive in another embodiment, of the development of a number of vascular complications in diabetic subjects. Specifically, subjects who are homozygous for the haptoglobin-1 allele are at decreased risk for developing retinopathy and nephropathy and conversely in one embodiment, those subjects exhibiting the haptoglobin-2 allele are at higher risk of developing diabetic nephropathy or retinopathy. This effect, at least for nephropathy, has been observed in both type 1 and type 2 diabetic subjects. In another embodiment, the haptoglobin phenotype is predictive of the development of macrovascular complications in the diabetic subject. In one embodiment, development of restenosis after percutaneous coronary angioplasty is significantly decreased in diabetic subjects with the 1-1 haptoglobin phenotype.

In one embodiment haptoglobin 2-2. phenotype is used as an independent risk factor, in relation to target organ damage in refractory essential hypertension, or in relation to atherosclerosis (in the general population) and acute myocardial infarction or in relation to mortality from HIV infection in other embodiments. In another embodiment, haptoglobin 2-2 phenotype make subjects more prone to oxidative stress, therefore, haptoglobin 2-2 phenotype is used in one embodiment as a negative predictor for cardiovascular disease in DM.

According to this aspect of the invention and in one embodiment, the invention provides a method of treating vascular complications in a subject carrying the Hp 2 allele, comprising reducing oxidative stress in said subject, wherein said subject is diabetic.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combination thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term. A method to treat diabetic cardiomyopathy may comprise in one embodiment, a method to reduce labile plasma iron in a diabetic patient, since the latter may lead to, or aggravate cardiomyopathy.

Patients having diabetes and having in one embodiment, an additional condition or disease such as cardiovascular disease, or ischemic heart disease, congestive heart failure, congestive heart failure but not having coronary arteriosclerosis, hypertension, diastolic blood pressure abnormalities, microvascular diabetic complications, abnormal left ventricular function, myocardial fibrosis, abnormal cardiac function, pulmonary congestion, small vessel disease, small vessel disease without atherosclerotic cardiovascular disease or luminal narrowing, coagulopathy, cardiac contusion, or having or at risk of having a myocardial infarction in other embodiments, are at particular risk for developing very serious cardiac insufficiencies including death because diabetic cardiomyopathy further adversely affects the subject's heart and cardiovascular system.

The term “preventing” refers in another embodiment, to preventing the onset of clinically evident pathologies associated with vascular complications altogether, or preventing the onset of a preclinically evident stage of pathologies associated with vascular complications in individuals at risk, which in one embodiment are subjects exhibiting the Hp-2 allele. In another embodiment, the determination of whether the subject carries the Hp-2 allele, or in one embodiment, which Hp allele, precedes the methods and administration of the compositions of the invention.

In another embodiment, the invention provides a method of reducing a myocardial infarct size resulting from ischemia-reperfusion injury in a subject carrying the Hp 2 allele, comprising reducing oxidative stress in said subject, wherein said subject is diabetic

In one embodiment, oxidative stress originating from Hp 2-1 or 2-2 phenotype leads to vascular complications in the general populations. It is also known that certain vascular complications are associated with oxidative stress associated with DM At present, however, it remains unclear, and cannot be predicted, whether Hp1-1 phenotype can affect the response to antioxidant supplementation for prevention of vascular complications in diabetic patients.

Haptoglobins contain both alpha chains and beta chains. Beta chains are identical in all haptoglobins, while alpha chains differ in one embodiment, between the two alleles of the haptoglobin gene The alpha 2 chain of haptoglobin is the result of a mutation based on an unequal crossing over and includes 142 amino acids, in contrast to the 83 amino acids of the alpha 1 chain. Immunologically the α-2 and α-2 chains are similar, with the exception of a unique sequence of amino acid residues in the α-2 chain (Ala-Val-Gly-Asp-Lys-Leu-Pro-Glu-Cys-Glu-Ala-Asp-Asp-Gly-Gln-Pro-Pro-Pro-Lys-Cys-Ile, SEQ ID NO:1)

In one embodiment, α-2 chain is represented by the sequence:

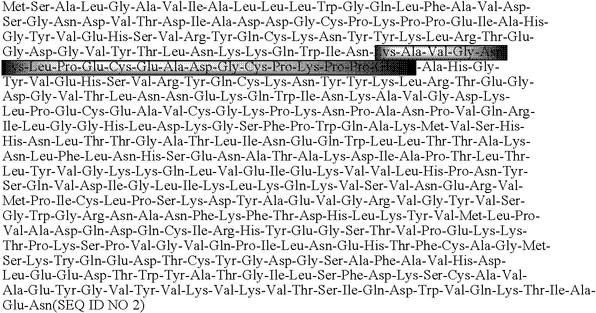

In one embodiment, hyperglycemia and the oxidative milieu created as a result of glucose autooxidation results in the formation of advanced glycation end-products (AGEs) or modified low density lipopioteins (ox-LDL) which can stimulate in another embodiment, the production of multiple inflammatory cytokines implicated in the pathological and morphological changes found in diabetic vascular disease. In one embodiment, vascular complications occur in diabetics over time, even though their blood sugar levels may be controlled by insulin or oral hypoglycaemics (blood glucose lowering) medications in another embodiment In one embodiment, diabetics are at risk of developing diabetic retinopathy, or diabetic cataracts and glaucoma, diabetic nephropathy, diabetic neuropathy, claudication, or gangrene, hyperlipidaemia or cardiovascular problems such as hypertension, atherosclerosis or coronary artery disease in other embodiments. In another embodiment, atherosclerosis causes angina or heart attacks, and is twice as common in people with diabetes than in those without diabetes. In one embodiment, the complications described hereinabove, are treated by methods and composition of th invention.

In one embodiment, antioxidant supplementation in diabetic patients homozygous for the haptoglobin 2 allele is beneficial in preventing adverse cardiovascular events.

In another embodiment, the vascular complication is a macrovascular complication such as chronic heart failure, cardiovascular death, stroke, myocardial infarction, coronary angioplasty associated restenosis, fewer coronary artery collateral blood vessels and myocardial ischemia in other embodiments. In one embodiment, the vascular complication is a microvascular complication, such as diabetic neuropathy, diabetic nepluopathy or diabetic retinopathy in other embodiment. In one embodiment, microvascular complications lead to renal failure, or peripheral arterial disease, or limb amputation in other embodiments.

Microvascular disease may be characterized in one embodiment, by an unevenly distributed thickening (or hyalinization) of the intima of small arterioles, due in another embodiment, to the accumulation of type IV collagen in the basement membrane, or microaneuxisyms of the arterioles, which compromises the extent of the maximal arteriolar dilation that can be achieved and impairs the delivery of nutrients and hormones to the tissues, or to remove waste in another embodiment. The vasculature distal to the arterioles may also be affected in one embodiment, such as by increased capillary basement membrane thickening, abnormalities in endothelial metabolism, or via impaired fibrinolysis, also resulting in reduced delivery of nutrients and hormones to the tissues, or waste removal in another embodiment. Microvascular disease results in one embodiment in microvascular diabetic complications, which in another embodiment, are treated by the methods of the invention.

In one embodiment, capillary occlusions constitute a characteristic pathologic feature in early diabetic retinopathy, and initiate neovascularization in another embodiment. Microaneurysms, intraretinal microvascular abnormalities and vasodilation are commonly found in early stages of diabetic retinopathy and have been correlated to capillary occlusions. IN another embodiment, leukocytes cause capillary obstruction that is involved in diabetic retinopathy. This obstruction is the result of the leukocytes' large cells volume and high cytoplasmic rigidity. Leukocytes can become trapped in capillaries under conditions of reduced perfusion pressure (e.g., caused by vasoconstriction) or in the presence of elevated adhesive stress between leukocytes and the endothelium, endothelial swelling, or narrowing of the capillary lumen by perivascular edema. Examples of leukocytes include granulocytes, lymphocytes, monocytes, neutrophils, eosinophils, and basophils. Elevated adhesive stress results in one embodiment, from release of chemotactic factors, or expression of adhesion molecules on leukocytes or endothelial cells in other embodiments.

Glucose combines in one embodiment, with many proteins in circulation and in tissues via a nonenzymatic, irreversible process to form advanced glycosylation end products (AGEs) The best known of these is glycosylated hemoglobin, a family of glucose-hemoglobin adducts. Hemoglobin A_{1c} (HbA_{1c}) is a specific member of this group and is useful in another embodiment, as an indicator of average glycemia during the months before measurement. Other AGEs are presumed to contribute to the complications of diabetes, such as glycosylated proteins of the basement membrane of the renal glomerulus. In one embodiment, candidate AGEs can be tested as biologically active agents according to the methods of this invention.

In one embodiment, retinal edema, hemorrhage, ischemia, microaneurysms, and neovascularization characterize diabetic retinopathy. In another embodiment advanced glycation end products (AGEs) cause the development of this complication. AGEs represent in one embodiment, an integrated measure of glucose exposure over time, ate increased in diabetic retina, and correlate with the onset and severity of diabetic retinopathy. In one embodiment, specific high affinity receptors bind AGEs and lead to the downstream production of reactive oxygen intermediates (ROI). ROIs are correlated in another embodiment, with diabetic retinopathy and increase retinal VEGF expression. The inhibition of endogenous AGEs in diabetic animals prevents in another embodiment, vascular leakage and the development of acellular capillaries and microaneurysms in the retina. Compounds capable of inhibiting endogenous AGEs are given in conjunction with the compositions of this invention as a part of a treatment according to the methods of the invention. In one embodiment, the compositions of the invention, comprising glutathion peroxidase or a biologically active analog thereof are used according to the methods of the invcention to treat diabetic retinopathy.

Diabetic Nephropathy refer in one embodiment, to any deleterious effect on kidney structure or function caused by diabetes mellitus (DM). Diabetic nephropathy progresses in one embodiment in stages, the first being that characterized by microalbuminuria. This may progress in another embodiment, to macroalbuminuria, or overt nepluopathy. In one embodiment, progressive renal functional decline characterized by decreased GFR results in clinical renal insufficiency and end-stage renal disease (ESRD).

The increase in renal mass associated with the Hp 2 allele in the diabetic state is explained in one embodiment, by the synergy between Hp-type dependent differences in the clearance of Hp-Hb complexes and the inability of Hp to prevent glycosylated Hb-induced oxidation. In another embodiment, since the Hp-glycosylated Hb complex is oxidatively active, it is of heightened importance in the diabetic subject to cleat the Hp-Hb complex as rapidly as possible. The Hp-2-2-Hb is cleared more slowly than Hp-1-1-Hb, thereby producing more oxidative stress in the tissues of Hp-2 carrying subjects. In one embodiment, the methods and compositions of the invention are used to treat diabetic nephropathy in subjects carlying the Hp-2 allele.

Diabetic neuropathy is the most common complication of diabetes mellitus (DM), in both types 1 and type 2 Diabetic neuropathy has been associated with a decrease in nerve conduction velocity, Na,K-ATPase activity and characteristic histological damage of the sciatic nerve. Of all complications of diabetes, neuropathy causes the greatest morbidity, and a decrease in the subject's quality of life. In one embodiment, development of secondary complications (eg, foot ulcers, cardiac arrhythmias) leads to amputations and death in patients with DM. Diabetic neuropathy is a heterogeneous syndrome affecting in another embodiment, different regions of the nervous system separately or in combination.

In one embodiment, the term “diabetic neuropathy” refers to a neuropathy caused by a chronic hyperglycemic condition. The diabetic neuropathy is classified in another embodiment, into groups of; multiple neuropathy, autonomic neuropathy and single neuropathy. Diabetic neurosis indicates in one embodiment, a symmetrical, distal, multiple neuropathy causing in another embodiment, sensory disturbance. Both multiple neuropathy and autonomic neuropathy are neuropathies characteristic of diabetics.

In one embodiment, complications arising out of microvascular disorders result in blood flow being disturbed by changes of the blood abnormalities (such as acceleration of platelet aggregation, increase of the blood viscosity and decrease of the red blood-cell deformity) or by changes of the blood vessel abnormalities (such as reduction of the production of nitric oxide from the endothelial cells of blood vessels and acceleration of the reactivity on vasoconstiictive substances), then the hypoxia of nerves is caused, and finally the nerves are degenerated. In another embodiment, when the platelet aggregation is accelerated by the chronic hyperglycemic state, the microvascular disturbance result in diabetic neuropathy

In another embodiment, Glutathione peroxidase, is an important defense mechanism against myocardial ischemia-reperfusion injury, and is markedly decreased in one embodiment, in the cellular environment of DM. In vitro and in vivo studies with BXI-51072 show in one embodiment, that glutahion peroxidase is capable of protecting cells against reactive oxygen species and in another embodiment, inhibiting inflammation via action as an inhibitor of NF-κB activation.

Glutathion peroxidase (GPX) can be found largely in mammals cells, in mitochondrial matrix and cytoplasm. It reacts in one embodiment, with a large number of hydroperoxides (R—OOH). Glutathion peroxidase is of great importance within cellular mechanism for detoxification, since it is able in another embodiment, to reduces, in the same manner, the hidroperoxides from lipidic peroxidation. GPX is distributed extensively in cell, blood, and tissues, and its activity decreases when an organism suffers from diseases such as diabetes. In one embodiment GPX is involved in many pathological conditions and is one of the most important antioxidant enzymes in living organisms However, the therapeutic usage of the native GPX is limited because of its instability, its limited availability, and the fact that is extremely difficult to prepare by using genetic engineering techniques because it contains selenocysteine encoded by the stop codon UGA.

Foul types of GPx have been identified: cellular GPx (cGPx), gastrointestinal GPx, extracellular GPx, and phospholipid hydroperoxide GPx cGPx, also termed in one embodiment, GPX1, is ubiquitously distributed. It reduces hydrogen peroxide as well as a wide range of organic peroxides derived from unsaturated fatty acids, nucleic acids, and other important biomolecules. At peroxide concentrations encountered under physiological conditions and in another embodiment, it is more active than catalase (which has a higher Kₘ. for hydrogen peroxide) and is active against organic peroxides in another embodiment. Thus, cGPx represents a major cellular defense against toxic oxidant species.

Peroxides, including hydrogen peroxide (H₂O₂), are one of the main reactive oxygen species (ROS) leading to oxidative stress. H₂O₂. is continuously generated by several enzymes (including superoxide dismutase, glucose oxidase, and monoamine oxidase) and must be degraded to prevent oxidative damage. The cytotoxic effect of H₂O₂ is thought to be caused by hydroxyl radicals generated from iron-catalyzed reactions, causing subsequent damage to DNA, proteins, and membrane lipids

NF-κB is a redox-sensitive factor that is activated in one embodiment, by the cytosolic release of the inhibitor κB (IκB) proteins and the ttanslocation of the active p50/p65 heterodimer to the nucleus. In another embodiment, increase in the production of radical oxygen species serves as a pathway to a wide variety of NF-κB inducers.

In one embodiment, administration of GPx or its pharmaceutically acceptable salt, its functional derivative, its synthetic analog or a combination thereof, is used in the methods and compositions of the invention.

In another embodiment haptoglobin phenotype influences the clinical course of atherosclerotic cardiovascular disease (CVD). In one embodiment, a graded risk of restenosis after percutaneous transluminal coronary artery angioplasty is related to the number of haptoglobin 2 alleles. In another embodiment diabetic individuals with the haptoglobin 2-1 phenotype are significantly more likely to have coronary artery collaterals as compared to individuals with haptoglobin 2-2 phenotype with a similar degree of coronary artery disease. Inter-individual differences in the extent of the coronary collateral circulation are the key determinant of the extent of a myocardial infarction in another embodiment. In another embodiment, diagnosis and selection of course of treatment according to the methods and compositions of the invention is preceded by the phenotypic determination of the Hp phenotype in the subject.

Cardiovascular disease (CVD) is the most frequent, severe and costly complication of type 2 diabetes. It is the leading cause of death among patients with type 2 diabetes regardless of diabetes duration. In one embodiment, allelic polymorphism contributes to the phenotypic expression of CVD in diabetic subjects. In another embodiment, the methods and compositions of the invention are used in the treatment of CVD in diabetic subjects.

The term “myocardial infarct” or “MI” refers in another embodiment, to any amount of myocardial necrosis caused by ischemia. In one embodiment, an individual who was formerly diagnosed as having severe, stable or unstable angina pectoris can be diagnosed as having had a small MI. In another embodiment, the term “myocardial infarct” refers to the death of a certain segment of the heart muscle (myocardium), which in one embodiment, is the result of a focal complete blockage in one of the main coronary arteries or a branch thereof. In one embodiment, subjects which were formerly diagnosed as having severe, stable or unstable angina pectoris, are treated according to the methods or in another embodiment with the compositions of the invention, upon determining these subjects cariy the Hp-2 allele and are diabetic.

The term “ischemia-reperfusion injury” refers in one embodiment to a list of events including: reperfusion arrhythmias, microvascular damage, reversible myocardial mechanical dysfunction, and cell death (due to apoptosis or necrosis). These events may occur in another embodiment, together or separately. Oxidative stress, intracellular calcium overload, neutrophil activation, and excessive intracellular osmotic load explain in one embodiment, the pathogenesis and the functional consequences of the inflammatory injury in the ischemic-reperfused myocardium. In another embodiment, a close relationship exists between reactive oxygen species and the mucosal inflammatory process.

In one embodiment haptoglobin protein impact the development of atherosclerosis. The major function of serum haptoglobin is to bind free hemoglobin, which in another embodiment, is thought to help scavenge labile plasma iron (LPI) and prevent its loss in the urine and to serve as an antioxidant thereby protecting tissues against hemoglobin mediated tissue oxidation. The antioxidant capacity of the different haptoglobin differ in one embodiment, with the haptoglobin 1-1 protein appealing to confer superior, antioxidant protection as compared to the other forms of the protein. Gross differences in size of the haptoglobin protein present in individuals with the different phenotypes explain in one embodiment, the apparent differences in the oxidative protection afforded by the different types of haptoglobin. Haptoglobin 1-1 is markedly smaller then haptoglobin 2-2 and thus more capable to sieve into the extravascular compartment and prevent in another embodiment, hemoglobin mediated tissue damage at sites of vascular injury. In one embodiment, the differences between the antioxidative efficiencies of the various Hp-phenotypes show the importance of determining the Hp phenotype being carried by the subject.

A major function of haptoglobin (Hp) is to bind hemoglobin (Hb) to form a stable Hp-Hb complex and thereby prevent Hb-induced oxidative tissue damage. Clearance of the Hp-Hb complex is mediated in one embodiment, by the monocyte/macrophage scavenger receptor CD163.

In another embodiment, the role of the Hp-Hb complex in modulating oxidative stiess and inflammation after ischemia-reperfusion is Hp genotype dependent. In one embodiment, Hp 2-Hb complexes are associated with increased Labile Plasma Iron (LPI), particularly in the diabetic state, resulting in another embodiment, in increased iron-induced oxidative injury in Hp 2 allele-cariying subjects. In one embodiment, specific receptors for LPI exist on cardiomyocytes through which LPI mediates its toxic effects.

In another embodiment, the production of I1-10 by the Hp-fib complex is Hp genotype dependent with markedly greater I1-10 production in Hp 1 mice after ischemia-reperfusion. I1-10. is an anti-inflammatory cytokine which inhibits NF-κB activation, oxidative stress and polymorphonuclear cell infiltration after ischemia-reperfusion

In one embodiment, interleukin 10 markedly attenuates ischemia-reperfusion injury by inhibiting NF-κB activation, or decreases oxidative stress and prevents polymorphonuclear cell infiltration in other embodiments. In another embodiment, Hp-Hb complex is formed early in the setting of an acute myocardial infarction secondary to hemolysis as evidenced by an acute fall in serum Hp levels. Hp 1-1-Hb complex induces in one embodiment, a marked increase in I1-10 release from macrophages in vitro acting via the CD163 receptor. In one embodiment, a Hp genotype dependent differences in I1-10 release exist in the PMBC's of a subject following non-lethal MI. In another embodiment, plasma levels of I1-10 in Hp 2 carrying subjects after ischemia-reperfision is not statistically significant from plasma levels of I1-10 in Hp 2 carlying subjects prior to ischemia-reperfusion.

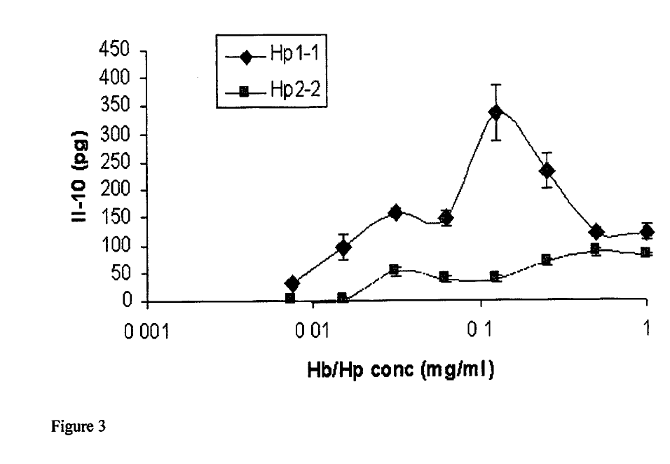

The normal concentration of the Hp-Hb complex in blood is 25 nM (5 ug/ml) at which no appreciable stimulation of I1-10 is observed with Hp 1-1 or Hp 2-2 (FIG. 3). In one embodiment, 150 nM Hp-Hb (50 ug/ml) which could readily be achieved following the hemolysis associated with reperfusion there is a significant increase in I1-10 release induced by Hp 1-1-Hb complexes as compared to Hp 2-2-Hb.

In one embodiment, compounds or methods leading to an increase in the amount of IL-10 released by cardiomycetes will cause a reduced MI, when in one embodiment they are given prior to or immideiately after MI.

In another embodiment, the invention provides a method of reducing a myocardial infarct size resulting from ischemia-reperfusion injury in a subject carTying the Hp 2. allele, comprising reducing oxidative stress in said subject, wherein said subject is diabetic, wherein the method, in another embodiment, further comprises administering to said subject an effective amount of glutathion peroxidase, its pharmaceutically accepted salt or a synthetic mimnetic thereof, which is in another embodiment benzisoselen-azoline or -azine derivatives or in another embodiment, is referred to as BXI-51072.

In one embodiment, the term BXI-51072, refers to benzisoselen-azoline or -azine derivatives represenetd by the following general formula:

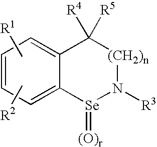

where: R¹, R²=hydrogen; lower alkyl; OR⁶; —(CH₂)m NR⁶R⁷; —(CH₂)_{q}NH₂; —(CH₂)ₘ NHSO₂ (CH₂)₂ NH_{2;} —NO₂; —CN; —SO₃H; —N⁺(R⁵)₂ O⁻; F; Cl; Br; I; —(CH₂)ₘ R⁸; —(CH₂)ₘ COR⁸; —S(O)NR⁶R⁷; —SO₂ NR⁶ R⁷; —CO(CH₂)ₚ COR⁸; R⁹; R³=hydrogen; lower alkyl; aralkyl; substituted aralkyl; —(CH₂)ₘ COR⁸; —(CH₂)_{q}R⁸; —CO(CH₂)ₚ COR⁸; ——(CH₂)ₘ SO₂ R⁸; —(CH₂)ₘ S(O)R⁸; R⁴=lower alkyl; aralkyl; substituted aralkyl; —(CH₂)ₚ COR⁸; —(CH₂)ₚR⁸; F; R⁵=lower alkyl;aralkyl; substituted aralkyl; R⁶=lower alkyl; aralkyl; substituted aralkyl; —(CH₂)ₘCOR⁸; —(CH₂)_{q}R⁸; R⁷=lower alkyl; aralkyl; substituted aralkyl; —(CH₂)ₘCOR⁸; R⁸=lower alkyl;aralkyl; substituted atalkyl; aryl; substituted axyl; heteroaryl; substituted heteroaryl; hydroxy;lower alkoxy; R⁹; R⁹=

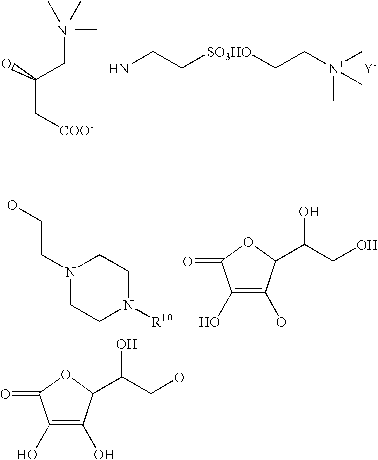

R¹⁰=hydrogen; lower alkyl;aralkyl or substituted aralkyl; atyl or substituted aryl; Y⁻ represents the anion of a pharmaceutically acceptable acid; n=0, 1; m=0, 1, 2; p=l, 2, 3; q=2, 3,4 and r=0, 1.

In another embodiment, BXI-51072 refers to benzoisoselen-azoline

In one embodiment, treating Hp 2 mice with the BXI-51072 have shown that BXI-51072. dramatically reduces MI size in this model. In another embodiment, Glutathione peroxidase, an important defense mechanism against myocardial ischemia-reperfusion injury, is markedly decreased in the environment of DM. In one embodiment, In vitro and in vivo tests with benzisoselen-azoline and -azine derivatives have shown that it is capable to protecting cells against reactive oxygen species and inhibiting inflammation in another embodiment, via its actions as a potent inhibitor of NF-κB activation.

In one embodiment, iron catalyzed reactions play a direct role in exacerbating ischemia reperfusion injury. In another embodiment, over 99% of iron carried in the plasma is bound to transferrin and is not redox active. LPI represents iron present in the plasma which is not bound to transferrin and which is highly redox active. An increased amount of LPI is generated in one embodiment from Hp 2-Hb complexes in the diabetic state.

In another embodiment, the invention provides a method of reducing a myocardial infarct size resulting from ischemia-reperfusion injury in a subject carrying the Hp 2 allele, comprising reducing oxidative stress in said subject, wherein said subject is diabetic, wherein the method, in another embodiment, further comprises reducing the level of labile plasma iron (LPI) below 0.3 μM.

When in one embodiment, iron transport proteins are overwhelmed, albeit transiently, the result, free iron in the circulation is termed labile protein iron (LPI) would be available to bind to other proteins with which it is not normally associated. This so-called labile iron may be taken up in another embodiment by a variety of tissues via secondary transport routes, with potential production of reactive oxygen species (ROS).

The traffic of nonheme iron, oxygen, and ascorbate in plasma, is in one embodiment, a potential source of reactive oxygen species (ROS) generated by teduction-oxidation cycling of iron via ascorbate and O₂ Such undesirable reactions are physiologically counteracted in another embodiment, by various protective molecules: transferin, the iron transport protein, which in another embodeiment, restricts iron's capacity for undergoing redox reactions; antioxidants such as glutathione in another embodiment, and ascorbate, which, together with iron, has the dual capacity of promoting redox cycling at relatively low concentrations and acting as a powerful scavenger of radical species at higher concentrations.

In another embodiment, LPI was found to be increased both in Hp 1 and Hp 2 DM mice after myocardial ischemia-reperfusion but that only in Hp 2 DM mice were LPI levels greater than 0.3 uM, the level of LPI associated in one embodiment, with myocardial toxicity (see e.g. Table 3).

In one embodiment, Hp 2 DM subjects have increased LPI as compared to Hp 1 DM subjects. In one embodiment following ischemia-reperfusion injury, with a rapid burst in Hp-Hb complex formation, there a significant increase in LPI in Hp 2. DM subjects. LPI is increased in another embodiment in both Hp 1 and Hp 2 DM subjects after myocardial ischemia-reperfusion. In another embodiment, only Hp 2 DM subjects exhibit LPI levels greater than 0.3 uM achieved, the level of LPI associated in one embodiment, with myocardial toxicity.

In one embodiment, the invention provides a method of reducing a myocardial infarct size resulting from ischemia-reperfusion injury in a subject carrying the Hp 2 allele, comprising reducing oxidative stress in said subject, wherein said subject is diabetic and wherein the method, in another embodiment, further comprises increasing the release of IL-10 in said subject.

In one embodiment, the production of I1-10 by the Hp-Hb complex is Hp genotype specific, with markedly greater I1-10 production in Hp 1. mice after ischemia-reperfusion. I1-10 is an anti-inflammatory cytokine which in another embodiment, inhibits NF-κB activation, or oxidative stress and polymotphonuclear cell infiltration after ischemia-reperfusion in other embodiments. I1-10 is critical in one embodiment, for the protection against reperfusion injury. The mechanism for myocardial protection provided in another embodiment by I1-10, is mediated in large part by the enzyme heme oxygenase. In one embodiment, I1-10 is a potent inducer of heme oxygenase. In another embodiment, heme oxygenase degrades cytosolic heme, generating CO and biliveidin, which are highly potent antioxidants and anti-inflammatory agents.

In one embodiment, IL-10 is an important mediator of monocytic deactivation, which in another embodiment inhibits the production of proinflammatoty cytokines [eg tumour necrosis factor (TNF)-α] and is a major depressor of antigen presentation and specific cellular immunity through the reduction of MHC class II antigen expression and IL-12 production in other embodiments.

In one embodiment increased redox active iron and decreased I1-10 in Hp 2 mice indicate an oxidative mechanism for the increased infarct size in these mice after ischemia-reperfusion injury.

In another embodiment, the invention provides a method of reducing a myocardial infarct by increasing the release of IL-10 in a subject, wherein increasing the release of IL-10 is done by administering to said subject an effective amount of Hp 1-1-Hb complex.

In one embodiment (see FIG. 3), stimulation of I1-10. in subjects cartyinh the Hp-2 allele occurs at concentrations of Hp-Hb that are readily achievable in vivo. The normal concentration of the Hp-Hb complex in blood is 25 nM (5 ug/ml) at which no appreciable stimulation of I1-10 is observed with Hp 1-1 or Hp 2-2 (FIG. 3). However, at 150 nM Hp-Hb (50 ug/ml) a significant increase in I1-10 release induced by Hp 1-1-Hb complexes as compared to Hp 2-2-Hb is evident.

In one embodiment, the Hp-1-1-Hb complex administered in the methods of this invention is between about 100 and about 150 nM, or in another embodiment, between about 150 and about 200 nM, or in another embodiment, between about 200 and about 250 nM, or in another embodiment, between about 250 and about 300 nM

In another embodiment, the invention provides a method of reducing a myocardial infarct by administrating to said subject an effective amount of IL-10.

In one embodiment, Hp genotype is a major determinant of morbidity and mortality in subjects with DM. The development of a model which anticipates the susceptibility conferred by the Hp genotype on diabetic complications allows in another embodiment, a detailed dissection of the molecular basis for this pathway and provide a platform on which rational therapies and drug design can be developed In one embodiment, the increased MI size associated with the Hp 2 allele in DM individuals may be attributed to increased oxidative stress and therefore strategies designed in another embodiment to decrease this oxidative stress provide significant myocardial protection.

Oxidative Stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS. In another embodiment, the methods and compositions of the invention are used in the treatment of complications or pathologies resulting from oxidative stress in diabetic subjects.

In another embodiment, the route of administration in the methods of the invention, using the compositions of the invention, is optimized for particular treatments regimens. If chronic treatment of vascular complications is required, in one embodiment, administration will be via continuous subcutaneous infusion, using in another embodiment, an external infusion pump. In another embodiment, if acute treatment of vascular complications is required, such as in one embodiment, in the case of miocardial infarct, then intravenous infusion is used.

According to this aspect of the invention and in one embodiment, the invention provides a method of assessing the risk of developing large size myocardial infarction following ischemia reperfusion injury in a diabetic subject, comprising analyzing the Hp phenotype in said subject, wherein Hp 2 allele indicates a high risk of developing increased size myocardial infarct (MI).

In one embodiment, the compositions of the invention described hereinbelow are used with the methods of the invention described above.

According to this aspect of the invention, and in another embodiment, the invention provides a composition for reducing the myocardial infarct in a diabetic subject carrying the Hp 2 allele, comprising in one embodiment glutathione peroxidase or an isomer, a functional derivative, a synthetic analog, a pharmaceutically acceptable salt or a combination thereof in other embodiments; and a pharmaceutically acceptable carrier, or excipient, flow agent, processing aid, a diluent or a combination thereof in other embodiments.

Biologically active derivatives or analogs of the proteins described herein include in one embodiment peptide mimetics. Peptide mimetics can be designed and produced by techniques known to those of skill in the art. (see e.g., U.S. Pat. Nos. 4,612,132; 5,643,873 and 5,654,276, the teachings of which are incorporated herein by reference). These mimetics can be based, for example, on the protein's specific amino acid sequence and maintain the relative position in space of the corresponding amino acid sequence. These peptide mimetics possess biological activity similar to the biological activity of the corresponding peptide compound, but possess a “biological advantage” over the corresponding amino acid sequence with respect to, in one embodiment, the following properties: solubility, stability and susceptibility to hydrolysis and proteolysis.

Methods for preparing peptide mimetics include modifing the N-terminal amino group, the C-terminal carboxyl group, and/or changing one or more of the amino linkages in the peptide to a non-amino linkage. Two or more such modifications can be coupled in one peptide mimetic molecule. Other forms of the proteins and polypeptides described herein and encompassed by the claimed invention, include in another embodiment, those which are “functionally equivalent.” In one embodiment, this term, refers to any nucleic acid sequence and its encoded amino acid which mimics the biological activity of the protein, or polypeptide or functional domains thereof in other embodiments.

In another embodiment, the invention provides a composition for reducing the myocardial infarct in a diabetic subject carrying the Hp 2. allele, comprising: BTX-51072 and a pharmaceutically acceptable carrier and a Hp-1-1-Hb complex in a concentration effective to increase release of IL-10. in said subject, or IL-10 in another embodiment, or a chelating agent capable of reducing labile plasma iron in said subject in another embodiment.

In one embodiment, the chelating agents used in the compositions of this invention, or methods of this invention are deferripione (L1), or EDIA in another embodiment, or ICL670 in another embodiment, or ascorbate in another embodiment, or a combination thereof in another embodiment.

In one embodiment, the invention provides a composition for reducing the myocardial infarct in a diabetic subject carrying the Hp 2 allele, comprising: BIX-51072 and a pharmaceutically acceptable carrier, or excipient, flow agent, processing aid, a diluent or a combination thereof in other embodiments wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In another embodiment, the composition further comprises a binder, a disintegrant, a buffer, a protease inhibitor, a surfactant, a solubilizing agent, a plasticizer, an emulsifier, a stabilizing agent, a viscosity increasing agent, a sweetner, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease inhibitors or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intraperitonealy, intraventricularly, or intracianially.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1 M and preferably 0.05 M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981;. Newmark et al, 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffeting agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free caiboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The active agent is administered in another embodiment, in a therapeutically effective amount. The actual amount administered, and the rate and time-course of administration, will depend in one embodiment, on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners Examples of techniques and protocols can be found in Remington's Pharmaceutical Sciences

Alternatively, targeting therapies may be used in another embodiment, to deliver the active agent more specifically to certain types of cell, by the use of targeting systems such as antibodies or cell specific ligands. Targeting may be desirable in one embodiment, for a variety of reasons, e.g. if the agent is unacceptably toxic, or if it would otherwise require too high a dosage, or if it would not otherwise be able to enter the target cells.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations

Controlled or sustained release compositions include formulation in lipophilic depots (e.g. fatty acids, waxes, oils). Also comprehended by the invention are particulate compositions coated with polymers (erg. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment; a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffet content (e.g., Tris-HCl, acetate, phosphate), pH and ionic strength, additives such as albumin or gelatin to prevent absorption to surfaces, detergents (e.g., Tween 20, Tween 80, Plutonic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheioplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease inhibitors, or permeation enhancers for various routes of administration, including parenteral, pulmonary, nasal, and oral.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include micropaiticles of poly(lactide-co-glycolide), polyacrylate, latex, starch, cellulose, dextran and the like Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duiation of release and the nature of the condition to be treated suppressed or inhibited

In one embodiment, the compositions of the invention ate administered in conunction with other therapeutica agents. Representative agents that can be used in combination with the compositions of the invention are agents used to treat diabetes such as insulin and insulin analogs (e.g. LysPro insulin); GLP-1 (7-37) (insulinotropin) and GLP-1 (7-36)-NH sub 2; biguanides: metformin, phenformin, buformin; .alpha 2-antagonists and imidazolines: midaglizole, isaglidole, deriglidole, idazoxan, efaroxan, fluparoxan; sulfonylureas and analogs: chlorpropamide, glibenclamide, tolbutamide, tolazamide, acetohexamide, glypizide, glimepiride, repaglinide, meglitinide; other insulin secretagogues: linogliride, A-4166; glitazones: ciglitazone, pioglitazone, englitazone, troglitazone, darglitazone, rosiglitazone; PPAR-gamma agonists; fatty acid oxidation inhibitors: clomoxir, etomoxir; .alpha-glucosidase inhibitors: acarbose, miglitol, emiglitate, voglibose, MDL-25,637, camiglibose, MDL-73,945; , beta-agonists: BRL 35135, BRL 37344, Ro 16-8714, ICI D7114, CL 316,243; phosphodiesterase inhibitors: L-386,398; lipid-loweling agents: benfluorex; antiobesity agents: fenfluramine; vanadate and vanadium complexes (e.g. Naglivan RTM )) and peroxovanadium complexes; amylin antagonists; glucagon antagonists; gluconeogenesis inhibitors; somatostatin analogs and antagonists; antilipolytic agents: nicotinic acid, acipimox, WAG 994 Also contemplated for use in combination with the compositions of the invention are pramlintide acetate (Symlin.™.), AC2993, glycogen phosphorylase inhibitor and nateglinide. Any combination of agents can be administered as described hereinabove.

In one embodiment, the term “polymorphism” refers to the occurrence of two or more genetically determined alternative sequences of alleles in a population A polymorphic marker or site is in another embodiment, the locus at which divergence occurs. In one embodiment, markers have at least two alleles, each occurring at frequency of greater than 1%, and in another embodiment, greater than 10% or 20% of a selected population. A polymorphic locus may in one embodiment be as small as one base pair. Polymorphic markers include in another embodiment, restriction fragment length polymorphisms, or variable number of tandem repeats (VNTR's), or hypervariable regions, or minisatellites, or dinucleotide repeats, or trinucleotide repeats, or tetranucleotide repeats, or simple sequence repeats, and insertion elements such as Alu. The first identified allelic form is in one embodiment, arbitrarily designated as the reference form and other allelic forms are designated as alternative or variant alleles. The allelic form occurring most frequently in a selected population is referred to in one embodiment, as the wildtype form. Diploid organisms are homozygous in one embodiment, or heterozygous for allelic forms in another embodiment. A dialleic or biallelic polymorphism has two forms. A tliallelic polymorphism has three forms.

In the practice of the methods of the present invention, an effective amount of compounds of the present invention of pharmaceutical compositions thereof, as defined above, are administered via any of the usual and acceptable methods known in the art, either singly or in combination with another compound or compounds of the present invention or other pharmaceutical agents, such as antibiotics, hormonal agents for the treatment of microvascular or macrovascular diseases such as insulin and so forth The method of administering the active ingredients of the present invention is not considered limited to any particular mode of administration. The administration can be conducted in one embodiment, in single unit dosage form with continuous therapy or in another embodiment, in single dose therapy ad libitum. Other modes of administration are effective for treating the conditions of retinopathy, nepluopathy or neuropathy. In other embodiments, the method of the present invention is practiced when relief of symptoms is specifically required, or, perhaps, imminent. The method hereof are usefully practiced in one embodiment, as a continuous or prophylactic treatment

Oxidative Stress refers in one embodiment to a loss of redox homeostasis (imbalance) with an excess of reactive oxidative species (ROS) by the singular process of oxidation. Both redox and oxidative stress are associated in another embodiment, with an impairment of antioxidant defensive capacity as well as an overproduction of ROS.

### EXAMPLES

### Example 1

### Haptoglobin Genotype Determines Myocardial Infarct Size in Diabetic Subjects

Materials and Methods

Animals

Wild type C57BL/6 mice carry only a class 1 Hp allele highly homologous to the human Hp 1 allele and are referred to as Hp 1 mice. The Hp 2 allele exists only in human. Mice containing the Hp 2 allele were generated by introducing the human Hp 2 allele as a transgene in a C57B1/6 Hp knockout genetic background.

Diabetes

Diabetes was induced by an intrapetitoneal injection of 200. mg/kg streptozotocin in 3 month old mice. The severity of diabetes was defined both by a spot non-fasting glucose (glucometer) and HbAlc (Helena Diagnostics). Myocardial infarction was produced 30-40 days after injection of streptozotocin.

Myocardial Ischemia-reperfusion Model

Myocardial injury was produced using a modification of a previously described ischemia-reperfusion model (Martire A, Fernandez B, Buehler A, Strohm C, Schaper J, Zimmermann R, Kolattukudy P E, Schaper W. Cardiac overexpression of monocyte chemoattractant protein-1 in transgenic mice mimics ischemic preconditioning through SAPK/JNK1/2 activation. Caidiovasc Res 57:523-534, 2003). Mice were anesthetized with a mixture of ketamine (150 mg/kg) and xylazine (9 mg/kg) and body temperature maintained at 37° C. using a heating pad. The trachea was intubated with a 21 G needle that was previously cut and had a blunt ending. The tube was connected to a respirator (Model 687, Harvard Apparatus). The respirator tidal volume was 1.2 ml/min and the rate was 100 strokes/min. A left lateral thoracotomy was made in the 4^{th} intercostal space, the skin, muscles and ribs were retracted and the pericaidial sac removed. Ligation of the left anterior descending coronary artery (LAD) was made using a 7/0 Ethicon virgin silk, non-absorbable suture, connected to a micro point reverse cutting 8. mm needle under vision with a stereoscopic zoom microscope (Nikon SMZ800). The LAD ligation was performed using an easily opened knot set on a PE50 silicon tube laying over the LAD. The ligation was released after 45 minutes followed by 1. hour of reperfusion. 15. min before the end of reperfusion interval, 0.5 cc of a 0.2% solution of propidium iodide (Sigma, Rehovot, Israel) was injected intraperitoneally. (Propidium iodide stains the nuclei of dead cells led when injected in vivo and as discussed below was used in this model to indicate infarcted myocardium). At the end of the reperfusion interval the LAD was re-occluded and a 4% solution of Thioflavin-S (Sigma) was injected into the ascending aorta. (Thioflavin stains endothelial cells blue when injected in vivo and was used in this model to indicate myocardium that was not at risk of myocardial infarction upon LAD ligation). The mice were then sacrificed, the right ventricle excised, and the left ventricle was cryopreserved with liquid nitrogen-cooled methylbutane.

Determination of Myocardial Infarct Size

The left ventricle was cut into 15 μm thick cryosections and every 20^{th} section was photographed using an inverted fluorescent Zeiss microscope, connected to a digital camera and a computer with quantitative ImagePro software (a total of 12 sections for each heart). The area at risk of MI upon LAD ligation was defined and measured as thioflavin negative (i.e., the non-blue stained area) The infarct area was defined as propidium positive regions (i.e. deep red).

Quantitation of infarct size and risk area was performed using an infarct analysis program with Matlab software, using pixel color coordinates (color intensity) for automated calculation of the ratios: infarct area/risk area (IA/RA), infarct area/left ventricle (IA/LV), risk area/left ventricle (RA/LV). All quantitation was performed by a single reader blinded to the diabetes status and Hp genotype of the preparations.

Administiation of BXI-51072 to Decrease Infarct Sizes

BXI-51072, a small molecular weight, orally bioavailable, catalytic mimic of glutathione peroxidase, was obtained from Oxis International (Portland, Oregon). BXI-51072 was prepared as a suspension in water at 1. mg/ml and was given by gastric lavage at a dose of 5 mg/kg (approximately 100 microliters) 30-40 minutes prior to LAD ligation.

Measurement of Labile Plasma Iron (LPI)

Heparinized plasma was collected from mice at the end of the reperfusion interval and was stored at −70° C. until assayed. Normally, more than 99% of plasma iron is found bound to transferrin and is neither chelatable nor redox active. Labile plasma iron (LPI) represents chelatable redox active iron in plasma which is not bound to transferrin. LPI was first described in individuals with iron overload disorders such as thalassemia and has been implicated in the cardiac disease associated with these disorders. LPI was measured as previously described using dihydrorhodamine (DHR) a sensitive fluorescent indicator of oxidative activity. In the assay to measure LPI each serum sample was tested under two different conditions: with 40 uM ascorbate alone and with 40 uM ascorbate in the presence of 50 uM iron chelator (deferiprone). The difference in the rate of oxidation of DHR in the presence and absence of chelator represents the component of plasma iron that is redox active For the assay, quadiuplicates of 20 ul of plasma were transferred to clear bottom 96 well plates. To two of the wells 180 ul of iron free Hepes-buffered saline containing 40 uM of ascorbate and 50 uM of the DHR was added. To the other two wells, 180 ul of the same solution containing the iron chelator (50 uM) was added Immediately following the addition of reagent, the kinetics of fluorescence increase were followed at 37° C. in a BMG. GalaxyFlouroStar microplate reader with a 485/538 nm excitation/emission filter pair, for 40 minutes, with readings every 2 minutes. The slopes of the DHR fluorescence intensity with time were then determined from measurements taken between 15-40 minutes. The LPI concentration (in uM) was determined from calibration curves relating the difference in slopes with and without chelator vs. Fe concentration. Calibration curves were obtained by spiking plasma-like media with Fe:nitrilotriacetic acid (NIA) to give a final concentration of 40-100 uM followed by serial dilution.

Measurement of I1-10 in the Plasma of Mice After Ischemia-reperfusion.

Plasma was collected from the mice as described above for LPI at the end of the reperfusion interval. An enzyme-linked immunoabsorbent assay (ELISA) was used to measure I1-10 (BioLegent, USA) according to the manufacturer's protocol. Measurements were performed on plasma samples diluted 1:12 in a 1% BSA solution in a final volume of 50 microliters. Recombinant murine I1-10 was used as a standard.

Stimulation of Human Peripheral Blood Derived Mononuclear Cells With Hp-Hb Complex and Measurement of Human IL-10 in the Conditioned Media.

Hp 1-1 and Hp 2-2 were purified by affinity chromatography from human serum. Hb was fleshly prepared from lysed red blood cells. Peripheral blood mononuclear cells (PBMCs) were isolated from whole blood with Histopaque-1077 solution (Sigma) and grown for 18 hours in 96 well plates in RPMI-1640 supplemented with 10% FBS and 40 ng/ml dexamethasone. These culture conditions have previously been demonstrated to induce maximal expression of the Hp-Hb receptor CD163 on PBMCs. After 18 hours, the cells were incubated with varying concentrations of the Hp-Hb complex (1:1 molar ratio) for different time intervals in order to define the dose-dependency and time course for the induction of I1-10. I1-10 was measured in the conditioned media of these cells using an ELISA for human I1-10 (Biosource, USA) without dilution. Recombinant human I1-10 was used as a standard

Statistical Analysis

Mice were segregated based on Hp genotype. Groups were compared for the measured parameters using student's t-test. All p values are two-sided and a p value of less than 0.05 was considered statistically significant.

Results

Baseline Characteristics of Mice.

There were no significant differences in the age, duration of diabetes, glucose or HbA1c levels between Hp 1 and Hp 2 diabetic mice (Table 1).

TABLE 1
Baseline characteristics of mice prior to MI segregated
by Hp genotype and DM status

| Hp | | | | | | |
|---|---|---|---|---|---|---|
| geno- | | | | DM | | |
| type | N | Weight | Age | duration | Glucose | HbA1c |
| Hp 1 | 8 | 22.0 ± 1.30 | 4.3 ± 0.30 | 40.1 ± 1.5 | 417 ± 45 | 13.1 ± 0.8 |
| Hp 2 | 7 | 22.8 ± 0.70 | 4.2 ± 0.10 | 34.0 ± 3.6 | 388 ± 62 | 13.6 ± 0.6 |

All data is presented as the average ± SME.
N, is the number of mice in each group.
Weight is in grams,
Age is in months,
DM duration in days,
glucose in mg/dl and
HbA1c is expressed as the percentage of total Hb.

Myocardial Infarction Size is Increased in Diabetic Hp 2 Mice.

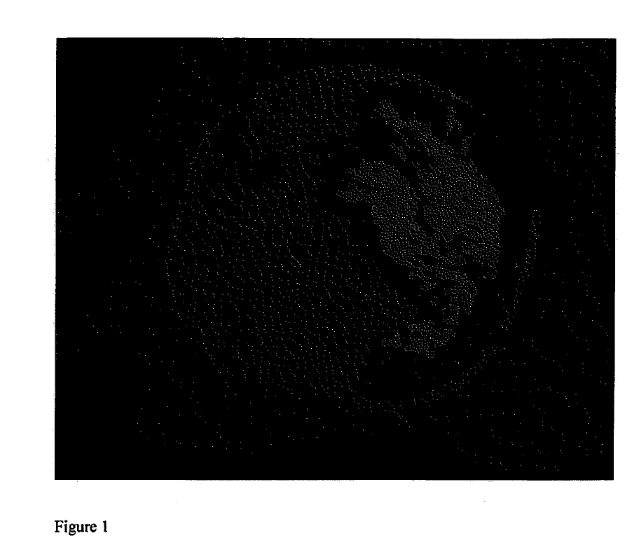

All mice were subjected to 45 minutes of LAD occlusion followed by 1 hour of reperfusion. Infarct area (IA) and the area at risk (RA) of MI were defied and calculated using propidium iodide and thioflavin as described in the Methods and as shown in FIG. 1. There was no significant difference in the area at risk of MI between Hp 1 and Hp 2 diabetic mice (Table 2). However, there was a statistically significant marked increase in infarct size (IA/RA) in Hp 2 mice compared to Hp 1 mice (44.3%+/−9.3% vs. 21.0+/−4.0%, n=7 and n=8 respectively, p=0.03) (Table 2)

TABLE 2
MI size is increased in Hp 2 mice

| Hp genotype | IA/RA (%) | IA/LV (%) | RA/LV (%) |
|---|---|---|---|
| Hp 1 | 21.0 ± 4.0 | 16.0 ± 3.5 | 74.2 ± 6.7 |
| Hp 2 | 44.3 ± 9.3 | 27.0 ± 3.3 | 70.2 ± 9.0 |

All data is presented as the average ± SME
IA, area of myocardial infarction.
RA, area at risk of MI with LAD occlusion.
LV, total left ventricular area.
There was a significant difference between DM Hp 2 and DM Hp 1 mice for IA/RA (p = 0.03) and for IA/LV (p = 0.04). There was no significant difference in RA/LV between Hp 1 and Hp 2 mice.

Labile Plasma Iron (IPI) is Increased in Diabetic Hp 2 Mice with MI.

Iron catalyzed reactions play a direct role in exacerbating ischemia reperfusion injury. However, over 99% of iron carried in the plasma is bound to transferrin and is not redox active. LPI represents iron present in the plasma which is not bound to transferrin and which is highly redox active. An increased amount of LPI is generated from Hp 2-Hb complexes under conditions which mimic the diabetic state In addition, Hp 2 DM mice have increased LPI as compared to Hp 1. DM mice, although the levels of LPI in these mice were less than 100 nM and of unknown significance. In the setting of ischemia-reperfusion with a rapid burst in Hp-Hb complex formation, it is assumed that there might be a significant increase in LPI in Hp 2 DM mice. LPI was found to be increased both in Hp 1 and Hp 2 DM mice after myocardial ischemia-reperfusion but that only in Hp 2 DM mice were LPI levels greater than 0.3. uM achieved, the level of LPI previously associated with myocardial toxicity (Table 3).

TABLE 3
LPI is increased and Il-10 is decreased in Hp 2 mice

| Haptoglobin genotype | LPI (uM) | Interleukin-10 (pg) |
|---|---|---|
| Hp 1 | 0.14 +/− 0.05 | 441 +/− 101 |
| Hp 2 | 0.45 +/− 0.11 | 62 +/− 51 |

LPI was measured in heparanized plasma collected at the end of the reperfusion interval as described in methods. LPI is in uM. There was a significant difference between LPI in Hp 1 and Hp 2 mice (n = 9 for each group, p = 0.02)

Interleukin-10 is Markedly Increased in Hp 1 DM Mice After Myocardial Ischemia and Reperfusion

Interleukin 10 markedly attenuates ischemia-reperfusion injury by inhibiting NF-κB activation, decreasing oxidative stress and preventing polymorphonuclear cell infiltration. Hp-Hb complex is formed early in the setting of an acute myocardial infarction secondary to hemolysis as evidenced by an acute fall in serum Hp levels. Hp 1-1-Hb complex induces a marked increase in I1-10 release from mactophages in vitro acting via the CD163 receptor. A Hp genotype dependent differences in I1-10 release may exist in the setting of MI. A highly significant increase in plasma levels of I1-10 in Hp 1 were found mice after myocaidial ischemia-reperfusion as compared to Hp 2 mice (Table 3) Notably, plasma levels of I1-10 found in Hp 2 mice after ischemia-reperfusion did not represent a statistically significant change from plasma levels of I1-10 found in Hp 2 mice prior to ischemia-reperfusion (Table 3)

I1-10 was measured as described in methods. Data for I1-10 represent the mean from 6 Hp 1 DM mice and 4 Hp 2 DM mice. There was a significantly greater increase in I1-10 production in Hp 1 DM mice as compared to Hp 2. DM mice after ischemia-reperfusion (p=0.01). Values of I1-10 shown represent the net increase in I1-10 obtained by subtraction of the values of I1-10 in the plasma of mice after ischemia-reperfusion from the values of I1-10 in the plasma of sham-treated mice (no coronary manipulation but otherwise treated identically). There was no difference in I1-10 plasma levels between Hp 1 and Hp 2 sham-treated mice with DM (mean 552±52 for Hp 1 and 466±28 for Hp 2, n=6). Moreover, values of I1-10 obtained in Hp 2 mice after ischemia-reperfusion did not represent a statistically significant change from the values of I1-10 obtained in Hp 2 sham-treated mice

Hp 1-1-Hb Complex Stimulates More I1-10 Release from Human PBMCs in vitro as Compared to the Hp 2-2-Hb Complex

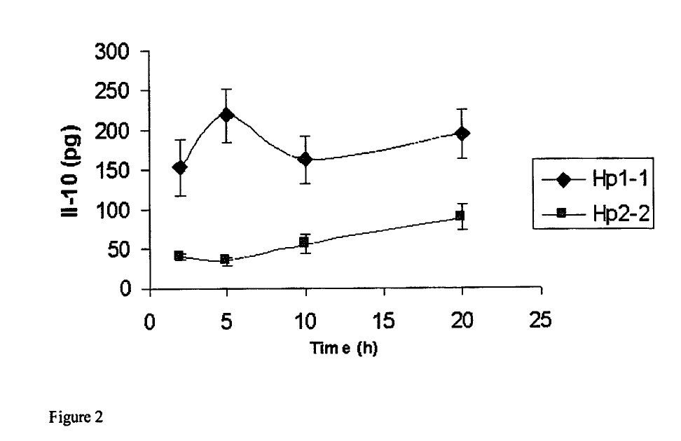

The Hp genotype-dependent differences in the induction of I1-10 in mice following ischemia-reperfusion described above was recreated in-vitro FIG. 2 demonstrates that within as little as 2 hours after stimulation there is significantly more release of I1-10 from PBMCs incubated with Hp 1-1-Hb as compared to Hp 2-2-Hb. Moreover, FIG. 3 demonstrates that stimulation of I1-10. in this system occurs at concentrations of Hp-Hb that are readily achievable in vivo. The normal concentration of the Hp-Hb complex in blood is 25 nM (5 ug/ml) at which no appreciable stimulation of I1-10 is observed with Hp 1-1 or Hp 2-2 (FIG. 3). However, at 150 nM Hp-Hb (50 ug/ml) which could readily be achieved following the hemolysis associated with reperfusion (50 ug of Hb corresponds to the amount of Hb released from less than 0.5 microliter of blood) there was a significant increase in I1-10 release induced by Hp 1-1-Hb complexes as compared to Hp 2-2-Hb.

Reduction in MI Size by Reducing Oxidative Stress.

The data with I1-10 and LPI indicates an oxidative mechanism to explain the more extensive myocardial infarction size in Hp 2. DM mice It is evident that intervention which decreased oxidative stress would provide significant protection to these Hp 2 carrying subjects. This was tested using the glutathione peroxidase mimic BXI-51072 given by gastric lavage to Hp 2 mice prior to ischemia-reperfusion injury. BXI-510.72 was found to dramatically reduced MI size (IA/RA) in this model (42.1+/−10.4% vs. 4.4+/−1.5%, p=0.0018) (Table 4).

TABLE 4
BTX-51072 decreases MI size in Hp 2 mice.

| Treatment | N | IA/RA (%) | IA/LV (%) | RA/LV (%) |
|---|---|---|---|---|
| BTX-51072 | 4 | 4.4 ± 1.5 | 3.4 ± 1.3 | 76.4 ± 6.5 |
| No BTX | 10 | 42.1 ± 10.4 | 25.3 ± 4.3 | 69.2 ± 8.7 |

All data is presented as the average ± SME.
IA, area of myocardial infarction.
RA, area at risk of MI with LAD occlusion.
LV, total left ventricular area.

Administration of BTX was by gastric lavage as described in methods. There was no significant difference in any parameter between mice which received gastric lavage with saline alone and mice which did not receive gastric lavage and therefore these two groups were pooled for the analysis described above There was a significant decrease in IA/RA (p=0.0018) and LA/LV (p=0 00015) between mice which did and did not receive BTX-51072. There was no significant difference between the two groups in the risk area.

The foregoing has been a description of certain non-limiting preferred embodiments of the invention. Those of ordinary skill in the art will appreciate that various changes and modifications to this description may be made without departing from the spirit or scope of the present invention, as defined in the following claims.

What is claimed is:

1. A method for treatment of a cardiovascular complication in a subject having the Hp-2 allele, comprising administering to said subject an effective amount of a compound, thereby reducing oxidative stress in said subject.

2. The method of claim 1, wherein said vascular complication is a myocardial infarct resulting from ischemia-reperfusion injury and wherein the treatment is reducing the size of said myocardial infarct (MI).

3. The method of claim 1, wherein said subject is diabetic.

4. The method of claim 1, wherein said treatment comprises treating, reducing incidence, or alleviating symptoms, eliminating recurrence, preventing recurrence, preventing incidence, improving symptoms, improving prognosis or combination thereof.

5. The method of claim 3, wherein said vascular complication is microvasculal complication or macrovascular complication.

6. The method of claim 5, wherein said macrovascular complication is a chronic heart failure, a cardiovascular death, a stroke, a myocardial infarction, a coronary angioplasty associated restenosis, a myocardial ischemia or a combination thereof.

7. The method of claim 5, wherein said microvascular complication is diabetic neuropathy, diabetic nephropathy or diabetic retinopathy.

8. The method of claim 1, wherein said compound is glutathione peroxidase, an isomer, a functional derivative, a synthetic analog, a pharmaceutically acceptable salt or a combination thereof.

9. The method of claim 1, preceded by determining the Hp phenotype in said subject.

10. The method of claim 1, comprising reducing the level of labile plasma iron (LPI) below 0.3 μM.

11. The method of claim 1, further comprising increasing the release of IL-10. in said subject.

12. The method of claim 11, wherein increasing the release of IL-10 is done by administrating to said subject an effective amount of Hp-1-1-Hb complex.

13. The method of claim 12, wherein said effective amount of Hp-1-1-Hb complex is between about 100 . to about 300 nM.

14. The method of claim 13, wherein said effective amount of Hp-1-1-Hb complex is about 150 nM.

15. The method of claim 3, comprising administering to said subject an effective amount of IL-10.

16. A method of assessing the risk of developing large size myocardial infarction following ischemia reperfusion injury in a diabetic subject, comprising analyzing the Hp phenotype in said subject, wherein Hp 2 allele indicates a high risk of developing increased size myocardial infarct (MI).

17. A composition for reducing the myocardial infarct in a diabetic subject carrying the Hp 2 allele, comprising: glutathione peroxidase or an analog thereof and a pharmaceutically acceptable carrier, excipient, flow agent, processing aid, a diluent or a combination thereof.

18. The composition of claim 17, further comprising Hp-1-1-Hb complex in a concentration effective to increase release of IL-10 in said subject.

19. The composition of claim 17, further comprising IL-10.

20. The composition of claim 17, further comprising a chelating agent capable of reducing labile plasma iron in said subject.

21. The composition of claim 20, wherein said chelating agent is deferriprone (L1), EDTA, ICL670, ascorbate or a combination thereof.

22. The composition of claim 17, wherein said carrier; excipient, lubricant, flow aid, processing aid or diluent is a gum, a starch, a sugar, a cellulosic material, an aclylate, calcium carbonate, magnesium oxide, talc, lactose monohydrate, magnesium stearate, colloidal silicone dioxide or mixtures thereof.

23. The composition of claim 17, comprising a binder, a disintegrant, a buffet, a protease inhibitor, a surfactant, a solubilizing agent, a plasticizer, an emulsifier, a stabilizing agent, a viscosity increasing agent, a sweetner, a film forming agent, or any combination thereof.

24. The composition of claim 17, wherein said composition is in the form of a pellet, a tablet, a capsule, a solution, a suspension, a dispersion, an emulsion, an elixir, a gel, an ointment, a cream, or a suppository.

25. The composition of claim 17, wherein said composition is in a form suitable for oral, intravenous, intraaorterial, intramuscular, subcutaneous, parentetal, transmucosal, transdermal, or topical administration.

26. The composition of claim 17, wherein said composition is a controlled release composition.

27. The composition of claim 17, wherein said composition is an immediate release composition.

28. The composition of claim 17, wherein said composition is a liquid dosage form.

29. The composition of claim 17, wherein said composition is a solid dosage form.


SEQUENCE LISTING (2 sequences)

SEQ ID NO: 1 - protein, 21 aa, Human
AVGDKLPECEADDGQPPPKCI

SEQ ID NO: 2 - protein, 406 aa, Human
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN